(19) United States
(12) Patent Application Publication
(10) Pub. No.: US 2003/0163143 A1
(43) Pub. Date: Aug. 28, 2003

(54) Apparatus and method for suturing in restricted space

(76) Inventor: Akio Wakabayashi, Newport Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/084,689

(22) Filed: Feb. 26, 2002

(51) Int. Cl. A61B 17/04

(52) U.S. Cl. 606/148

(57) ABSTRACT

Apparatus and methods of the present invention make possible suturing procedures in restricted spaces. In one embodiment, an applicator operated substantially perpendicular to a surgical field permits single-handed operation of a suture assembly to position a suture hook through tissues to be sutured. The suture hook having suture attached resists withdrawal of the suture from the tissues. A clip applicator, also operated substantially perpendicular to the surgical field, permits single-handed application of a suture clip which grips the suture to form a stitch which maintains required pressure on the tissues. In end-to-side anastomosis and like procedures, a tubular arteriotomy tool inserted in a graft provides a hook to secure tissue that is severed by rotating a sharpened edge of the tool against the tissue, the removal of the tissue facilitated by the hook.

### BACKGROUND OF THE INVENTION

1. Field of the Invention

The present invention relates to suturing and, more particularly, to apparatus and methods for performing surgical suturing in restricted spaces.

2. Description of the Related Art

It is common in surgical procedures to use sutures to attach one piece of soft tissue to another. Traditional suturing tools include straight suturing needles and curved suturing needles. Straight suturing needles are typically handled directly by the surgeon's hands. Curved suturing needles are typically manipulated using a needle driver that grips the needle at a middle point thereof. The curved suturing needle is driven through tissue by rotating the needle driver about its longitudinal axis.

Traditional suturing techniques generally require an open field of access to the tissue to be sutured so that the surgeon's hands or the needle driver can be positioned immediately adjacent the tissue. When sutures are required to be placed in narrow or deep locations, traditional suturing techniques are difficult to perform because of the limited space available for maneuvering the suturing instruments. In such cases, surgeons typically must make long incisions and create wide surgical fields within the patient's body to accommodate the instruments. This results in greater patient discomfort after surgery and increased recovery times.

In certain cases, suturing in narrow or deep locations can be performed endoscopically. However, existing endoscopic techniques generally require the surgeon to manipulate multiple tools, repeatedly releasing and re-gripping them. Such techniques are tedious, time consuming, and are not suitable in many situations.

### SUMMARY OF THE INVENTION

The present invention overcomes the problems of the prior art by providing suturing devices and methods for suturing of soft tissue to make such suturing possible in restricted spaces.

In one embodiment, the present invention is a suturing device. The suturing device comprises (1) a sheath having a proximal end and a distal end, the distal end configured to enter soft tissue when moved in a first direction; (2) a sheath handle at the proximal end of the sheath; (3) a suture slidably disposed within the sheath and having a proximal end and a distal end; (4) a suture handle at the proximal end of the suture, the suture handle configured to move the suture within the sheath when moved relative to the sheath handle; and (5) a suture hook at the distal end of the suture, the suture hook within the sheath when the suture handle is located a first distance from the sheath handle, the suture hook expelled from the sheath when the suture handle is positioned to a second distance from the sheath handle, the suture hook deploying upon the expelling to resist movement of the suture in a second direction substantially opposite the first direction. In accordance with certain aspects of this embodiment, the suture hook is a J-hook suture hook or a T-bar suture hook, and the suture hook may be integrally formed with the distal end of the suture or may be selectively attached. In accordance with other aspects of this embodiment, the suture may be a wire suture, a braided suture or a monofilament suture. In a preferred aspect, the suturing device further comprises a suture applicator having first and second applicator members, the suture applicator configured to move the suture handle relative to the suture sheath handle when the first and second applicator members are moved relative to each other. Preferred aspects of this embodiment are ones wherein the suture applicator is operable with one hand, the suture applicator facilitates the expelling of the suture hook and the first and second directions are substantially perpendicular to a surgical field, or a combination of those. In other preferred aspects, the embodiment further comprises a flange on a distal end of the suture applicator to regulate a penetration of the sheath within the soft tissue, or a lock substantially preventing movement of the suture sheath handle relative to the suture handle, or both.

In another embodiment, the present invention is a suture clip applicator. The suture clip applicator comprises: (1) a shaft with a handle; (2) a triggering member; and (3) a clip wedge that moves relative to the shaft and dislodges from a suture clip upon receiving a signal from the triggering member.

In still another embodiment, the present invention is a suture clip applicator. The suture clip applicator comprises: (1) a shaft having a clip retention member; (2) a lever movably coupled to the shaft; and (3) a clip wedge linked to the lever so that the clip wedge moves relative to the clip retention member when the lever is moved. In a preferred aspect, the suture clip applicator further comprises a suture clip having two wings and a wedge bore, the wings biased toward each other, the suture clip configured to receive the clip wedge in the wedge bore to separate the wings by a distance greater than a suture diameter, the clip retention member configured to hold the suture clip substantially stationary while the clip wedge moves, the holding of the suture clip causing the clip wedge to be withdrawn from the wedge bore whereupon the wings move toward each other. A preferred aspect of this embodiment is one wherein the suture clip applicator positions the suture clip to a position along a suture wherein, prior to the withdrawing of the clip wedge, the suture is between the wings and the suture clip is proximate a soft tissue undergoing suturing, and wherein the shaft is substantially perpendicular to a surgical site during the positioning and during the withdrawing of the clip wedge. A further preferred aspect is one wherein the suture clip applicator is adapted so that one hand can perform both the positioning and the withdrawing. An alternative preferred aspect of this embodiment is one wherein the suture clip is configured so that the wings grip a suture between them with a first force sufficient to maintain the grip in the presence of a second force applied to the suture clip in a direction along the suture, the second force substantially the same as that required to hold two to tissues together in a successful surgical suturing procedure.

In a further embodiment, the present invention is a suture clip applicator. The suture clip applicator comprises: (1) an applicator frame having an applicator shaft; (2) an applicator handle connected to the applicator shaft near a handle end of the applicator shaft; (3) a shoulder connected to the applicator shaft near a clip end of the applicator shaft; (4) a lever movably coupled to the applicator frame; (5) a retractor coupled to the lever, the retractor configured to move from an apply position into a retract position in a path substantially along the applicator shaft when the lever is moved relative to the applicator handle; (6) a clip wedge connected to a clip end of the retractor; and (7) a suture clip having opposing wings biased to exert a force toward each other, the clip wedge fitted into a wedge bore of the suture clip when the retractor is in the apply position, the fitting of the clip wedge creating a distance between the wings that is greater than the diameter of a suture, the shoulder preventing the suture clip from travelling with the clip wedge in the path when the lever is moved relative to the applicator handle, the movement of the lever thus removing the clip wedge from the wedge bore and causing the wings of the suture clip to spring together.

In yet another embodiment, the present invention is a method for suturing. The method comprises the steps of: (1) advancing a suture assembly into a surgical field in an operating direction substantially perpendicular to the surgical field until a distal end of a suture sheath of the suture assembly penetrates tissues to be sutured together; (2) moving a suture within the suture sheath such that a suture hook on a distal end of the suture emerges from the distal end of the suture sheath and deploys to resist withdrawal of the suture from the penetrated tissues; and (3) removing the suture assembly from the surgical field in a direction substantially opposite the operating direction while the suture hook remains deployed with the suture attached thereto. A preferred aspect of this embodiment comprises the further steps of: (4) advancing a suture clip applicator into the surgical field in the operating direction; (5) positioning a suture clip carried by the suture clip applicator about the suture at a position along the suture appropriate to create a pressure upon the tissues at least sufficient to surgically join the tissues; (6) releasing the suture clip such that it grips the suture to maintain the pressure; and (7) withdrawing the suture clip applicator from the surgical field in a direction substantially opposite the operating direction. An advantageous aspect of this embodiment is one wherein multiple stitches of a surgical suturing procedure are placed in tissues by repeating steps (1) through (7). An alternative advantageous aspect of this embodiment comprises the further steps of placing a clip wedge between two wings of the suture clip prior to the positioning of the suture clip and removing the clip wedge during the releasing of the suture clip. A preferred aspect of the method comprising steps (1) to (3) is one wherein the advancing of the suture assembly and the moving of the suture within the suture sheath are both performed by operating a suture applicator with a single hand, the suture applicator carrying the suture assembly.

Yet another embodiment of the present invention is a method for suture clipping. The method comprises the steps of: (1) advancing a suture clip applicator into a surgical field in an operating direction substantially perpendicular to the surgical field; (2) positioning a suture clip carried by the suture clip applicator about the suture at a position along the suture appropriate to create a pressure at least sufficient to surgically join the tissues; and (3) releasing the suture clip such that it grips the suture to maintain the pressure. A preferred aspect of this embodiment comprises the further steps of placing a clip wedge between two wings of the suture clip prior to the positioning of the suture clip and removing the clip wedge during the releasing of the suture clip. An advantageous aspect of this embodiment is one wherein the advancing, the positioning, the removing and the releasing are all performed by operating the suture clip applicator with one hand.

A still further embodiment of the present invention is an arteriotomy tool. The arteriotomy tool comprises: (1) a tubular body, the distal end of the tubular body having a sharpened edge to cut through and sever tissue upon rotation of the tubular body, and (2) a hook provided at the distal end of the tubular body to hold the severed tissue. A preferred aspect of this embodiment is one wherein the hook is provided through a guide channel within the tubular body.

### BRIEF DESCRIPTION OF THE DRAWINGS

FIG. 1A illustrates a traditional end-to-end anastomosis;

FIG. 1B illustrates a traditional end-to-side anastomosis;

FIG. 1C illustrates the rotational movement of a conventional needle driver;

FIG. 2A illustrates a representation of a suturing device in accordance with aspects of one embodiment of the present invention having a suture hook in an undeployed position;

FIG. 2B is a cross-sectional view of the suture hook in the undeployed position;

FIG. 2C illustrates a representation of the suturing device with the suture hook in a deployed position;

FIG. 2D is a cross-sectional view of the suture hook in the deployed position;

FIG. 3A is an enlarged view of a T-bar suture hook in an undeployed position;

FIG. 3B is an enlarged view of the T-bar suture hook in a deployed position;

FIG. 4A illustrates a representation of a suturing device in accordance with aspects of an embodiment of the present invention having a suture hook in an undeployed position;

FIG. 4B is an enlarged view representing the suture hook in the deployed position;

FIG. 4C illustrates a representation of a suturing device with a suture hook in a deployed position;

FIG. 4D illustrates a representation of an embodiment of a safety lock in accordance with the present invention;

FIG. 5A is an enlarged view of a portion of a suture handle of the suturing device of FIG. 4A;

FIG. 5B is a longitudinal cross-sectional view of a suture sheath of the suturing device of FIG. 4A;

FIG. 6 is a side elevational view of a suture applicator in accordance with aspects of an embodiment of the present invention;

FIG. 7 is a rear elevational view of the suture applicator of FIG. 6, with a cover of the applicator in an open position;

FIG. 8A is a rear elevational view of the suture applicator of FIG. 6 with the cover of the applicator in a closed position;

FIG. 8B is a front elevational view of a distal tip of the suture applicator of FIG. 6;

FIG. 9A is a top plan view of a suture clip in accordance with aspects of an embodiment of the present invention, illustrating the suture clip in a closed position;

FIG. 9B is a top plan view of the suture clip of FIG. 9A in an open position;

FIG. 10 is a side elevational view of a suture clip applicator in accordance with aspects of an embodiment of the present invention;

FIG. 11A is a horizontal cross-sectional view of the suture clip applicator of FIG. 10;

FIG. 11B is a vertical cross-sectional view of the suture clip applicator of FIG. 10;

FIG. 12A illustrates a method of perpendicular suturing in accordance with aspects of the present invention;

FIG. 12B is an enlarged view of the distal tip of a suturing device in accordance with aspects of an embodiment of the present invention;

FIG. 12C illustrates a method of perpendicular suturing in accordance with aspects of the present invention;

FIG. 12D is an enlarged view of the distal tip of a suturing device in accordance with aspects of an embodiment of the present invention;

FIG. 13A illustrates an end-to-side vascular anastomosis performed in accordance with one embodiment of the present invention;

FIG. 13B is a cross-sectional view of the anastomosis of FIG. 13A;

FIG. 14A illustrates a representation of an arteriotomy tool in accordance with aspects of an embodiment of the present invention;

FIG. 14B illustrates a lateral cross-sectional view of an arteriotomy tool in accordance with aspects of an embodiment of the present invention;

FIG. 14C illustrates a frontal cross-sectional view of the arteriotomy tool illustrated in FIG. 14B; and

FIG. 14D illustrates a representation of another arteriotomy tool in accordance with aspects of an embodiment of the present invention.

### DETAILED DESCRIPTION

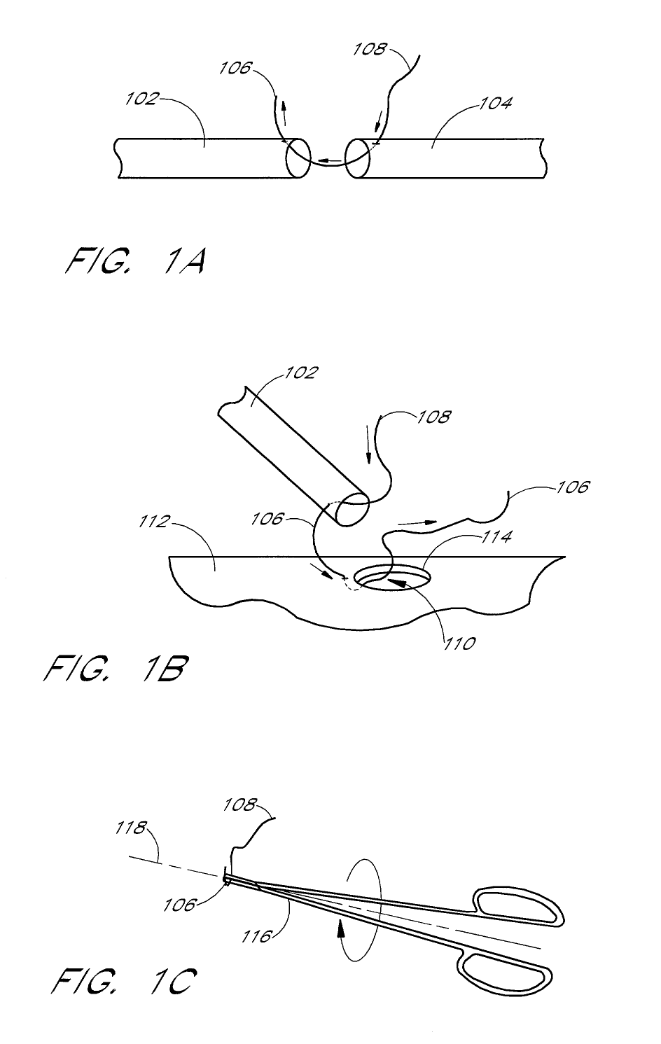

FIG. 1A illustrates a traditional end-to-end anastomosis. In the traditional procedure, a first artery 102 and a second artery 104 are anastomosed with a curved needle 106 and suture 108.

FIG. 1B illustrates a traditional end-to-side anastomosis. The artery 102 is anastomosed to an arteriotomy 110 of a second artery 112 using a curved needle 106 and suture 108. Typically, the second artery 112 is clamped on both sides of the anastomotic line 114 before the procedure begins. An arteriotomy 110 is then performed along an anastomotic line 114, after which the first artery 102 is sutured to the second artery 112 along the anastomotic line 114 at the location of the arteriotomy 110.

FIG. 1C illustrates the rotational motion of a conventional needle holder 116. The curved needle 106 with suture 108 is held by the needle holder 116 so that the plane of travel of the curved needle 106 is perpendicular to the longitudinal axis 118 of the needle holder 116. The needle holder 116 is then rotated about its longitudinal axis 118 to drive the needle 106 through the tissue to be sutured.

A surgeon grips the needle holder 116 by placing one or more fingers through one oval ring of the needle holder 116 and by placing a thumb through the other oval ring of the needle holder 116. The surgeon holds the curved needle 106 firm at the tip of the needle holder 116 by exerting opposing force with the thumb and fingers and then sutures tissues by using hand, wrist and/or forearm movements to rotate the curved needle 106 in a generally circular path to thread suture 108 through the tissues. Because, during suturing, the axis of rotation of the needle holder 116 is generally parallel to the surgical field and to the surfaces of tissues being sutured, a wide and open surgical field is required to accommodate the manipulation required for suturing.

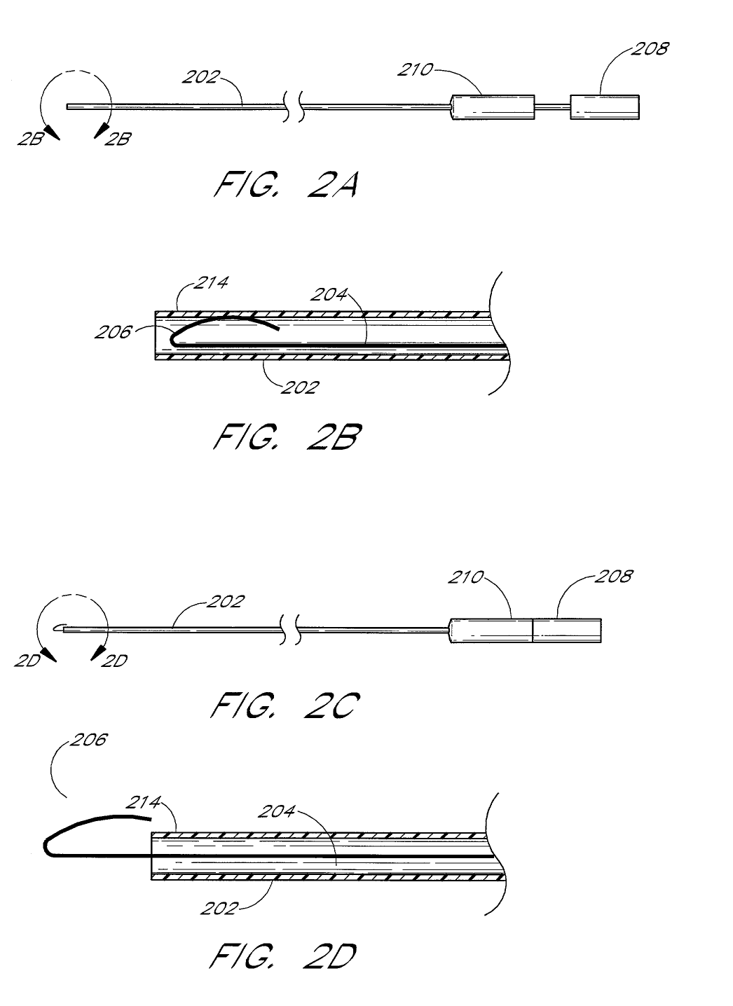

A first embodiment of a suturing device having certain features and advantages in accordance with the present invention is illustrated in FIG. 2A. In the illustrated embodiment, the suturing device comprises a suture sheath 202 connected to a suture sheath handle 210. In one embodiment, the suture sheath handle 210 has flattened surfaces, thus having a rectangular cross section. In other embodiments, the suture sheath handle 210 may have flattened surfaces with roughly triangular or other multi-sided cross section or may be cylindrical with a circular or oval cross section.

Disposed within the suture sheath 202 is a metal wire suture 204 (see FIG. 2B) having a J-hook suture hook 206 (see FIG. 2B) at a distal end thereof. A suture handle 208 is connected to the suture 204 at its proximal end. Like the suture sheath handle, 210, the suture handle 208 may have flattened surfaces with a rectangular cross section, or it may have a triangular or other multi-sided cross section, or it may be cylindrical with a circular or oval cross section.

The suture 204 and J-hook suture hook 206 slide within the suture sheath 202 as the suture handle 208 is moved toward or away from the suture sheath handle 210. It will be appreciated by those of ordinary skill that the suture need not be wire suture 204, but could be made from any other suitable material of sufficient rigidity to slide within the suture sheath 202. Generally, the closer the inner diameter of the suture sheath 202 to the outer diameter of the suture 204, the less suture rigidity will be needed for the suture 204 to slide within the suture sheath 202 upon relative movement of the suture handle 208 and suture sheath handle 210. In a preferred embodiment, the J-hook suture hook 206 is made of thin wire using a metal such as stainless steel. As with the suture 204, it will be appreciated that the J-hook suture hook 206 could be formed using other materials.

In one embodiment, the suture sheath 202 is approximately 0.4 mm in outer diameter, approximately 0.05 mm in thickness and approximately 25 cm in length. In such embodiment, the outer diameter of the suture 204 is smaller than 0.3 mm. It will be appreciated by those of ordinary skill that suture sheaths 202 of larger or smaller dimensions may be used along with sutures 204 of appropriately matching dimension.

FIG. 2B is an enlarged view of the J-hook suture hook 206 disposed inside the suture sheath 202. In the illustrated embodiment, the suture sheath 202 has a sharpened tip 214 to penetrate a tissue, such as a blood vessel (not shown). The term tissue, as used herein, refers to both natural bodily tissues and synthetic tissues, such as, for example, certain polymers. When the suture handle 208 is separated from the suture sheath handle 210 (as shown in FIG. 2A), the J-hook suture hook 206 resides inside the tip of the suture sheath 202, as illustrated in FIG. 2B.

FIG. 2C illustrates the suturing device with the J-hook suture hook 206 in a deployed position and with the suture handle 208 moved to rest against the suture sheath handle 210. Such advancement of the suture handle 208 toward the suture sheath handle 210 advances the suture 204 through the suture sheath 202. When the suture handle 208 abuts the suture sheath handle 210, as illustrated in FIG. 2C, the J-hook suture hook 206 is outside the tip of the suture sheath 202.

FIG. 2D is an enlarged cross-sectional view of the tip of the suture sheath 202 showing the J-hook suture hook 206 in its deployed state. The material from which the J-hook suture hook 206 is made has memory properties such that when the J-hook suture hook 206 is bent to a shape other than its deployed state, it will, upon release, spring back to its original deployed shape and will not remain in the shape to which it was bent. While resting in the tip of the suture sheath 202, the J-hook suture hook 206 is bent to fit within the passage defined by the inner wall of the suture sheath 202, and the inner wall thus prevents the J-hook suture hook 206 from expanding to its deployed shape. When the suture handle 208 is advanced to meet the suture sheath handle 210, the J-hook suture hook 206 is forced out of the suture sheath 202, and, upon exit from the suture sheath 202, the J-hook suture hook 206 springs into its deployed shape, as shown in FIG. 2D.

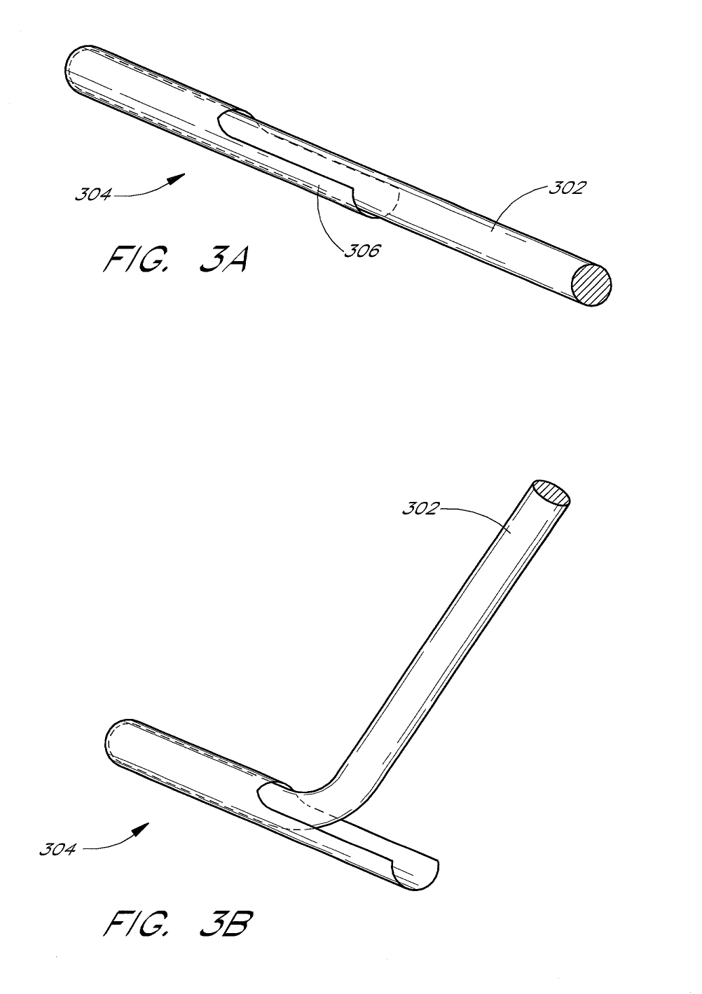

Another suture hook and suture suitable for use in the present invention is illustrated in FIGS. 3A and 3B. FIG. 3A is an enlarged view of a flexible polymer suture 302 and a T-bar suture hook 304. In the illustrated embodiment, the distal portion of the T-bar suture hook 304 comprises a thin-walled hollow cylinder. The proximal portion of the T-bar suture hook 304 comprises a semi-cylindrical trough 306. The thin-walled hollow cylinder and adjoining semi-cylindrical trough 306 are preferably made from a continuous piece of rigid material such as stainless steel or other material not toxic to natural bodily tissue. The suture 302 is threaded through the semi-cylindrical trough portion and into the thin-walled cylindrical portion of the T-bar suture hook 304 and is then attached to the distal end of the T-bar suture hook 304 with epoxy or cement, or by tying a knot in the distal end of suture 302, or by other suitable means. Prior to use, the T-bar suture hook 304 and suture 302 reside in the suture sheath 202 and the suture 302 extends through and is cradled by the semi-cylindrical trough portion of the suture hook 304. While undeployed within the suture sheath, the T-bar suture hook 304 and suture 302 are thus substantially coaxial.

FIG. 3B illustrates the T-bar suture hook 304 in an open or deployed position. Unconstrained by external means such as the suture sheath 202, the T-bar suture hook 304 and suture 302 no longer remain coaxial, but return to an approximately T-shape configuration. The suture 302 no longer extends through the semi-cylindrical trough portion of the T-bar suture hook 304.

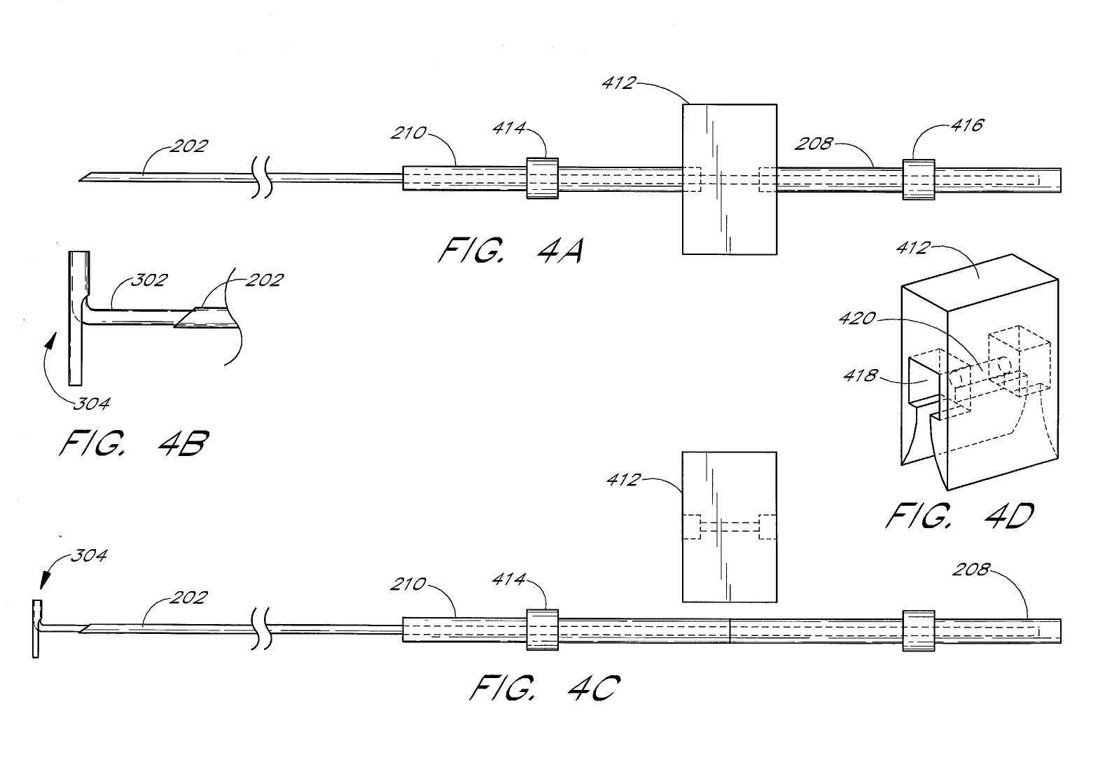

A second embodiment of a suturing device having features in accordance with the present invention is illustrated in FIG. 4A. In the illustrated embodiment, the suturing device comprises a suture sheath 202 connected to a suture sheath handle 210. A suture handle 208 is connected to the suture 302 (see FIG. 4B) at a proximal end thereof. The suture handle 208 is separated from the suture sheath handle 210 by a safety lock 412. With the safety lock 412 in place, the suture handle 208 may not be advanced toward the suture sheath handle 210, and the suture hook remains undeployed inside the tip of the suture sheath 202. Thus, the safety lock 412 prevents any accidental or undesirable deployment of the suture hook, whether it is a T-bar suture hook 304 or a J-hook suture hook 206.

Accordingly, as used in an operation, the suture sheath 202 is advanced to penetrate tissue, such as, for example, an arterial wall (not shown). Once the suture sheath 202 has penetrated tissue, the safety lock 412 is removed from between the suture sheath handle 210 and suture handle 208, and the suture handle is advanced toward the suture sheath handle 210 as illustrated in FIG. 4C.

In a preferred embodiment, the width of the safety lock is just slightly longer than distance a J-hook suture hook 206 or a T-bar suture hook 304 must travel to emerge from the tip of the suture sheath 202 and thereupon deploy. Thus, when the handles 210 and 208 meet, the T-bar (or other) suture hook 304 is pushed out of the suture sheath 202. Once deployed, either the T-bar suture hook 304 or the J-hook suture hook 206 open to resist being drawn out of the tissue.

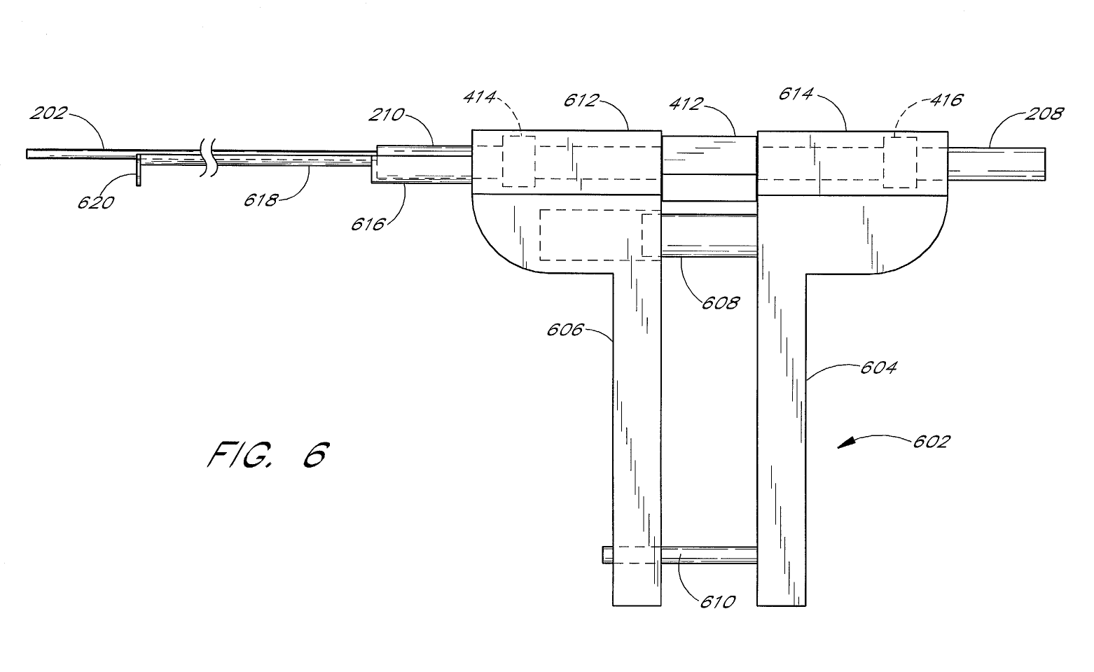

As further illustrated in FIGS. 4A and 4C, the suture sheath handle 210 and the suture handle 208 have respective flanges 414 and 416. The flanges 414 and 416 facilitate positioning the suturing assembly in an applicator (see FIGS. 6-8).

FIG. 4D illustrates a representation of a safety lock 412 in accordance with one embodiment of the present invention. The safety lock 412 is machined from a pliable, resilient plastic, which is slotted to clip over ends of the suture sheath handle 210 and the suture handle 208 nearest each other. Rectangular channels 418 formed part way into the safety lock 412 receive ends of the handles 210, 208. A narrow cylindrical channel 420 formed through the safety lock 412 receives the suture extending between ends of the handles 210, 208. The slot in the safety lock 412 runs from its bottom through and past the rectangular and cylindrical channels stopping short of the top of the safety lock 412. The slot permits generous separation, with modest force, of two halves of the safety lock 412. When the ends of the handles 210, 208 have been fit into the two larger rectangular channels 418 and the suture has been fit into the narrow cylindrical channel 420, separation pressure is removed from two halves of the safety lock 412 to create a gripping force upon the ends of the handles 210, 208, thus preventing unwanted movement of the handles 210, 208 with respect to each other when the suturing device is not in operation.

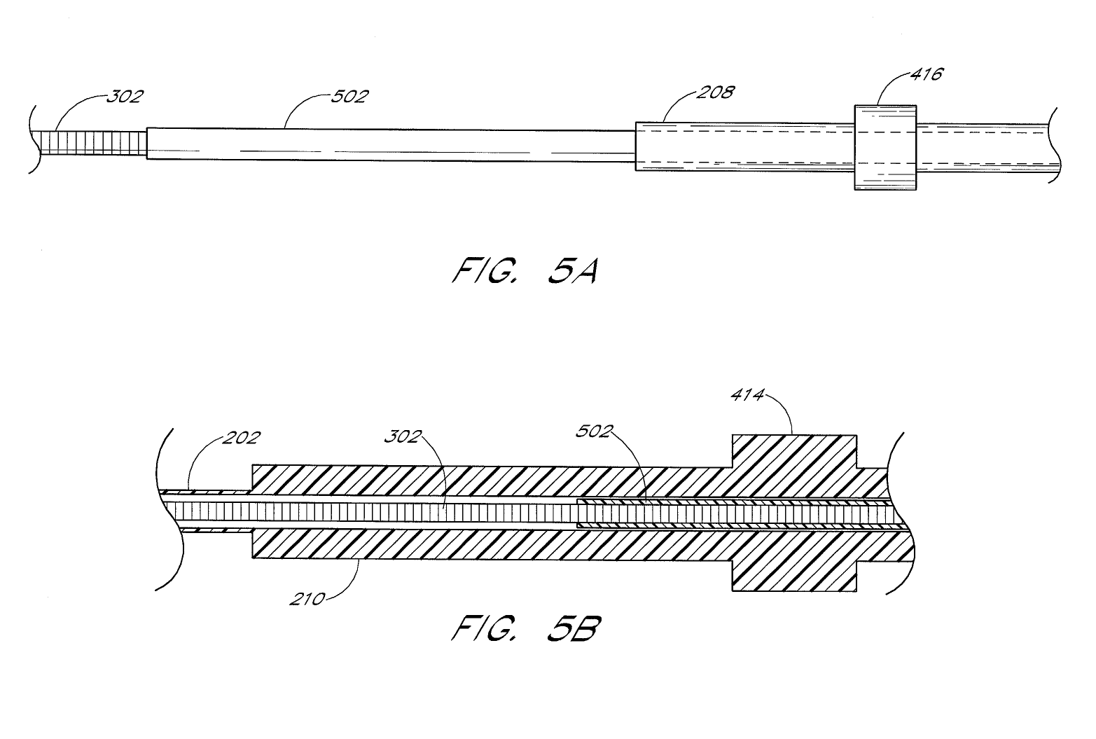

FIG. 5A is an enlarged view of a suture handle 208 in accordance with an embodiment of the present invention. A suture cover 502 is provided between the suture handle 208 and the suture 302. The suture cover 502 preferably comprises a thin-walled metal tube that provides a rigid means for advancing the flexible suture 302 into the suture sheath handle 210 and prevents kinking of the flexible suture 302.

FIG. 5B is a longitudinal cross-sectional view of a portion of a suturing device in accordance with an embodiment of the present invention. As illustrated in FIG. 5B, the suture cover 502 extends into the suture sheath handle 210, while the suture 302 extends through the suture sheath handle 210.

One embodiment of an applicator 602 for use with the suturing device is illustrated in FIG. 6. In the illustrated embodiment, the applicator 602 has two substantially parallel handles 604 and 606 that support the suture handle 208 and the suture sheath handle 210. A pair of posts 608 and 610 are fixed to the proximal handle 604 and are received into bores (not shown) formed in the distal handle 606. Both handles 604 and 606 have covers 612 and 614 to constrain the suture handle 208 and suture sheath handle 210.

The distal handle 606 has a suture sheath handle support 616 and a suture sheath support 618. A depth guard flange 620 is provided at the distal end of the suture sheath support 618 and is used to control the depth of penetration of the suture sheath 202 into the tissue to be sutured. The suture sheath 202 is supported by the suture sheath support 618. The safety lock 412 is interposed between the distal handle 606 and proximal handle 604 of the suture applicator 602.

In operation, a surgeon may grip the applicator 602 about both handles 604 and 606 using one hand. The surgeon may use the applicator 602 to guide and advance the suture sheath 202 toward and into the tissue to be sutured. Once the tissue has been penetrated, the safety lock 412 is removed and the surgeon may squeeze together the applicator handles 604 and 606 to thereby force the suture hook (either T-bar or J-hook or possibly other suture hook) out of the suture sheath and into a deployed position, such as behind an arterial wall.

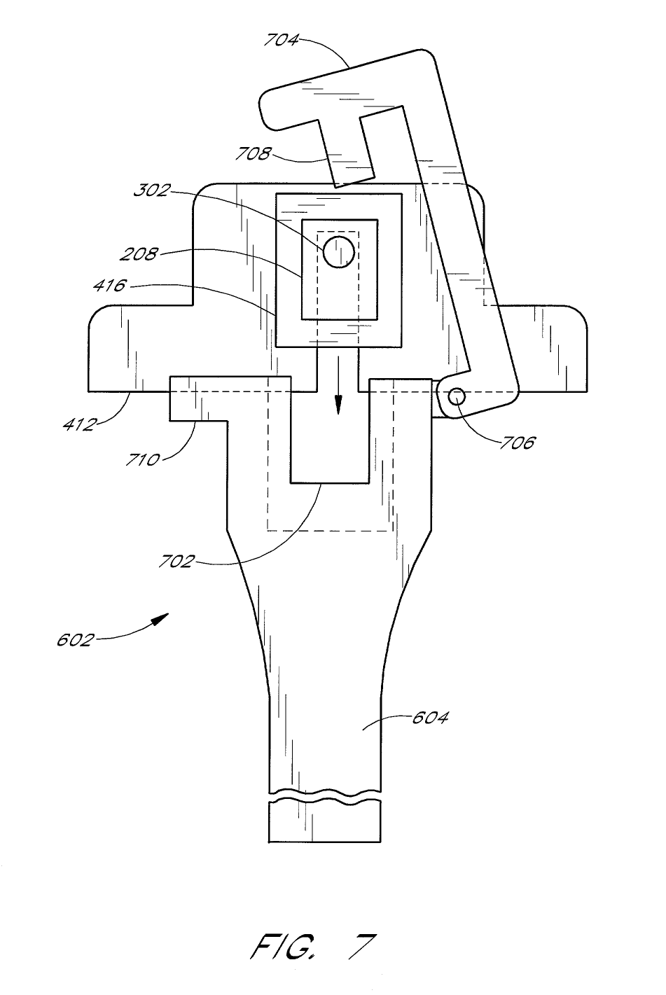

FIG. 7 illustrates a rear view of the suture applicator 602 in an unlocked or open position. The suture handle 208 and flange 416 fit snugly inside a groove 702 of the proximal suture applicator handle 604. A cover 704 rotates about a pin 706 that is fixed to the suture applicator handle 604.

A second embodiment of a safety lock for use with the applicator 602 is illustrated in FIG. 7. The safety lock 412 of FIG. 7 is shaped roughly archlike and fits over the top of the applicator 602 between the suture sheath handle 210 and the suture handle 208. The safety lock 412 may be removed to activate the applicator for use by lifting the safety lock upward and off of the suture assembly.

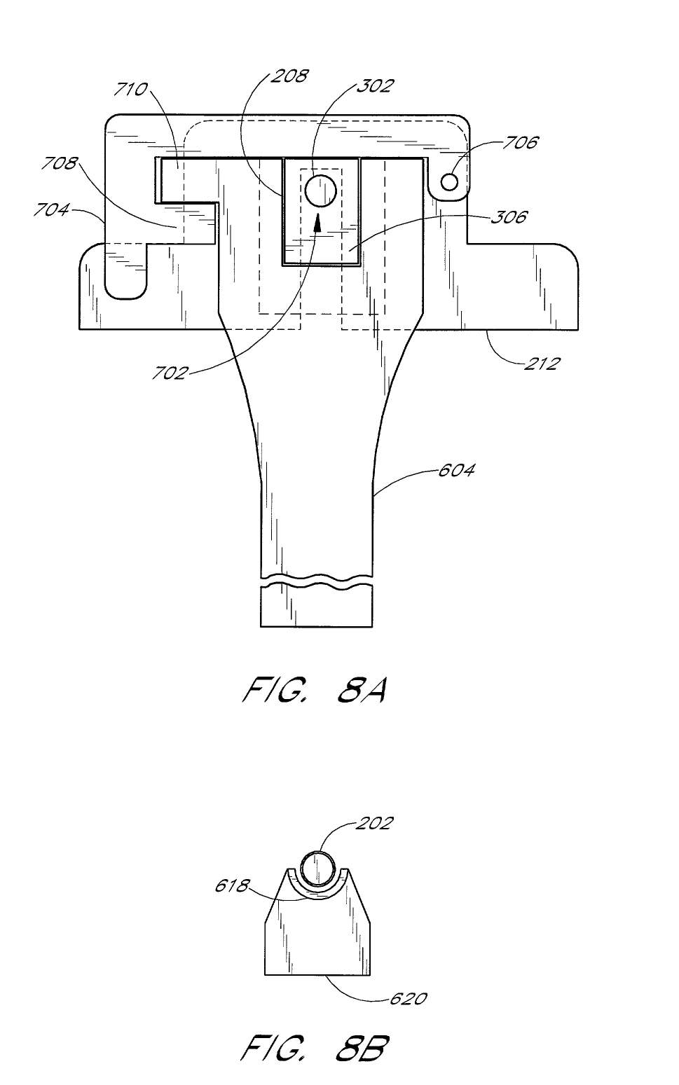

FIG. 8A illustrates a rear view of the suture applicator 602 in a locked or closed position. As illustrated in both FIGS. 7 and 8A, the cover 704 has a latch member 708 that cooperates with a latch member 710 of the proximal handle 604 of the applicator 602 to hold the suture handle 208 in place.

FIG. 8B is an enlarged view of the distal tip of the suture applicator 602. The suture sheath 202 is supported by the suture sheath support 618. The depth flange 620 is fixed to the end of said suture sheath support 618.

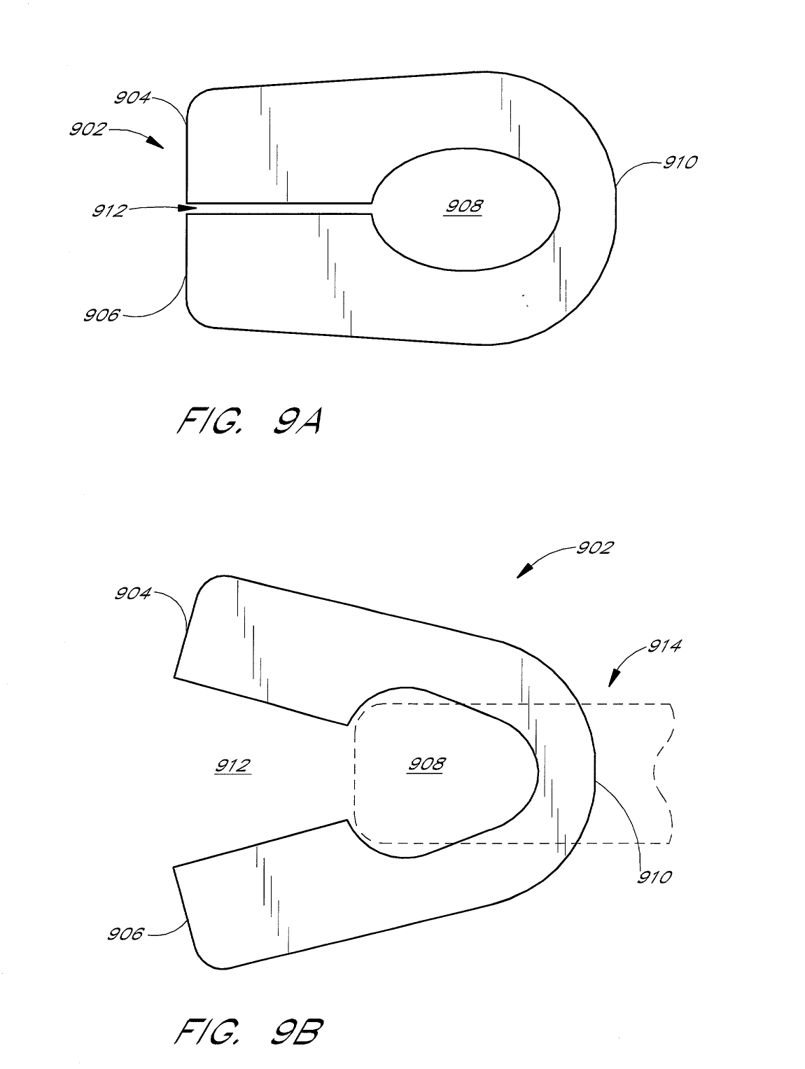

One embodiment of a suture clip having certain features in accordance with the present invention is illustrated in FIG. 9A. In FIG. 9A, the suture clip 902 is in a closed position. In the closed position, the suture clip 902 generally has a U-shape. The clip 902 comprises two wings 904 and 906 separated by an oval bore 908. A space 912 is formed between the wings 904 and 906 having a width less than a diameter of the suture 302.

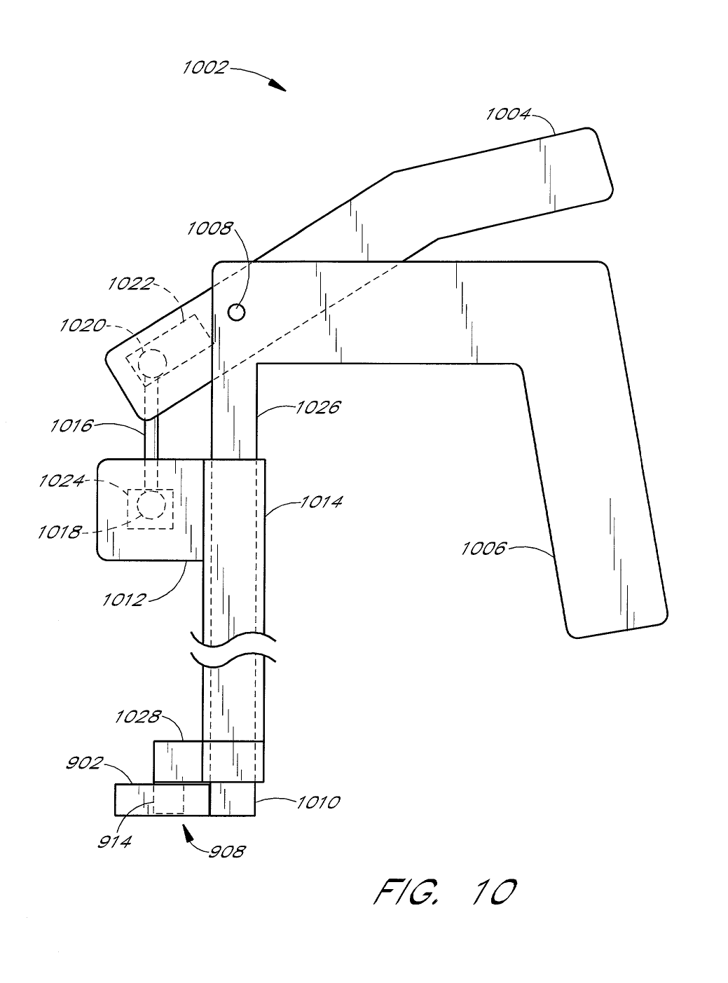

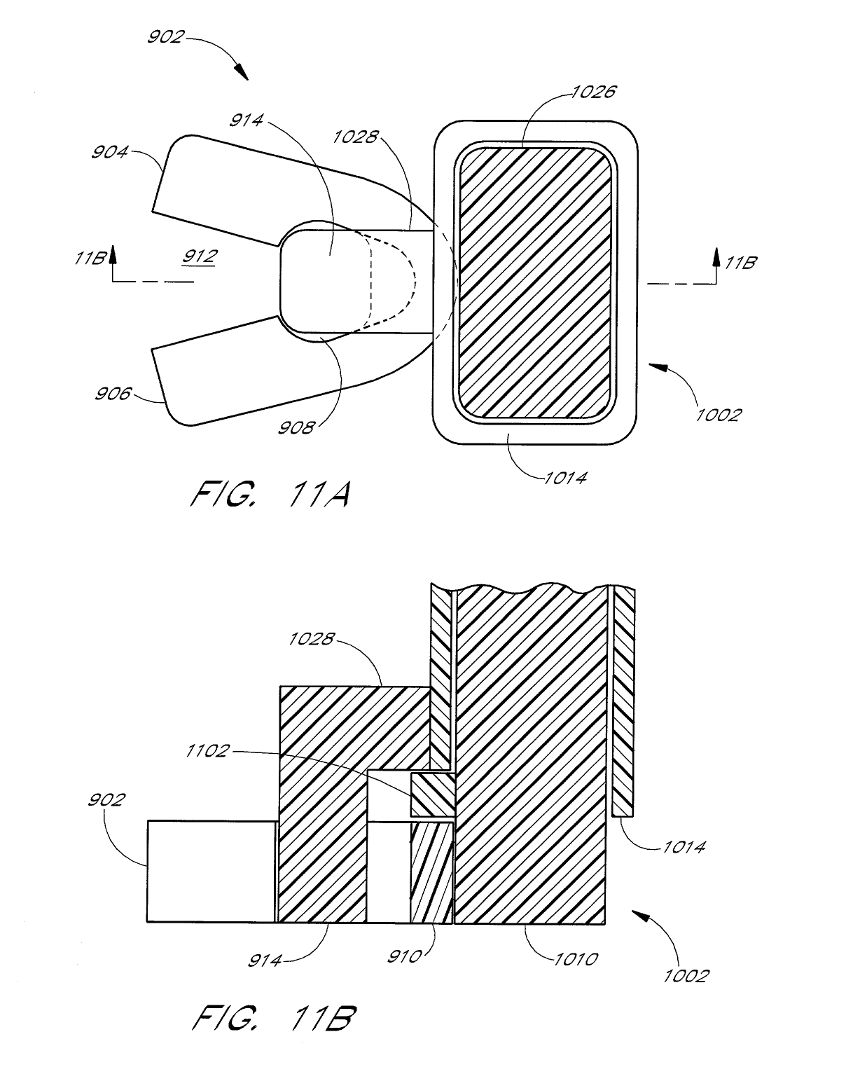

FIG. 9B illustrates the suture clip 902 in an open position. Prior to application, the suture clip 902 is forced to remain in the open position by a wedge 914 (see FIGS. 10, 11A and 11B). The widened space 912 between the two opposing wings 904 and 906 in this open position allows an easy placement of the suture 302 between the open wings 904 and 906. When the wedge 914 is removed from the oval bore 908, the wings 904 and 906 resiliently close to frictionally grasp the suture 302. Accordingly, the suture clip 902 is made from a material having elastic memory properties which return the clip to its deployed or closed state when a deforming pressure, such as that caused by the wedge 914, is removed.

One embodiment of a clip applicator 1002 having certain features in accordance with the present invention is illustrated in FIG. 10. In that embodiment, a lever 1004 is pivotally attached to a handle 1006 of the applicator 1002 by a pin 1008. The lever 1004 is connected to an upper horizontal extension 1012 of a sliding retractor 1014 by a bar 1016 with ball ends 1018 and 1020. The ball ends 1018 and 1020 are able to move freely inside spaces 1022 and 1024 of the lever 1004 and sliding retractor 1014.

The sliding retractor 1014 preferably is a tubular structure that is slidably positioned over a distal portion 1026 (shaft) of the handle 1006. When the lever 1004 is actuated by pushing it towards the handle 1006, the double ball-end bar 1016 pulls the upper horizontal extension 1012 of the sliding retractor 1014. This causes the sliding retractor 1014 to move proximally along the distal handle portion 1026. A lower horizontal extension 1028 and the wedge 914 move relative to the distal handle section 1026, thus dislodging the wedge 914 from the suture clip bore 908. When the wedge 914 is dislodged from the wedge bore 908, the clip 902 resiliently springs into the deployed or closed position illustrated in FIG. 9A. It will be appreciated by those of ordinary skill in mechanical design that alternative mechanisms activated by a lever or another type of triggering member, such as a trigger or a button, could be used to move the sliding retractor 1014 in a direction along the distal handle portion 1026. In particular, it will be understood to those of ordinary skill that designs based upon magnetic, electric, hydraulic and/or pneumatic principles are readily applicable, any of which could receive a signal from a triggering member and responsively move the sliding retractor 1014 to dislodge the wedge 914 from the suture clip 902.

As illustrated in FIG. 11A, the wedge 914 forces open the wings 904 and 906 of the suture clip 902 creating the space 912. The wedge 914 preferably is an integral part of the lower horizontal extension 1028 of the sliding retractor 1014 that slides over the shaft 1026.

As illustrated in FIG. 11B, a shoulder 1102 of the shaft 1010 prevents the proximal clip end 910 from moving with the wedge 914 and lower horizontal extension 1028 as the wedge 914 is dislodged from the suture clip 902. The shoulder 1102 holds the proximal end 910 of the suture clip 902 down while the wedge 914 is dislodged and the clip 902 closes about the suture.

An alternative clip may be used in accordance with the present invention, such as, for example, a crimp clip having two wings that are crimped together with a pincer-type applicator. More particularly, a crimp clip placed in a pincer member of a pincer-type applicator is positioned about a suture with the suture between the wings of the crimp clip. Activation of a trigger member of the pincer-type applicator causes the pincer member to squeeze the wings of the crimp clip together at a point along the suture where the clip resists pressure from the sutured tissues. The pincer-type applicator is operated single-handedly substantially perpendicular to the surgical field, with the pincer member attached to the distal end of a shaft of the pincer-type applicator.

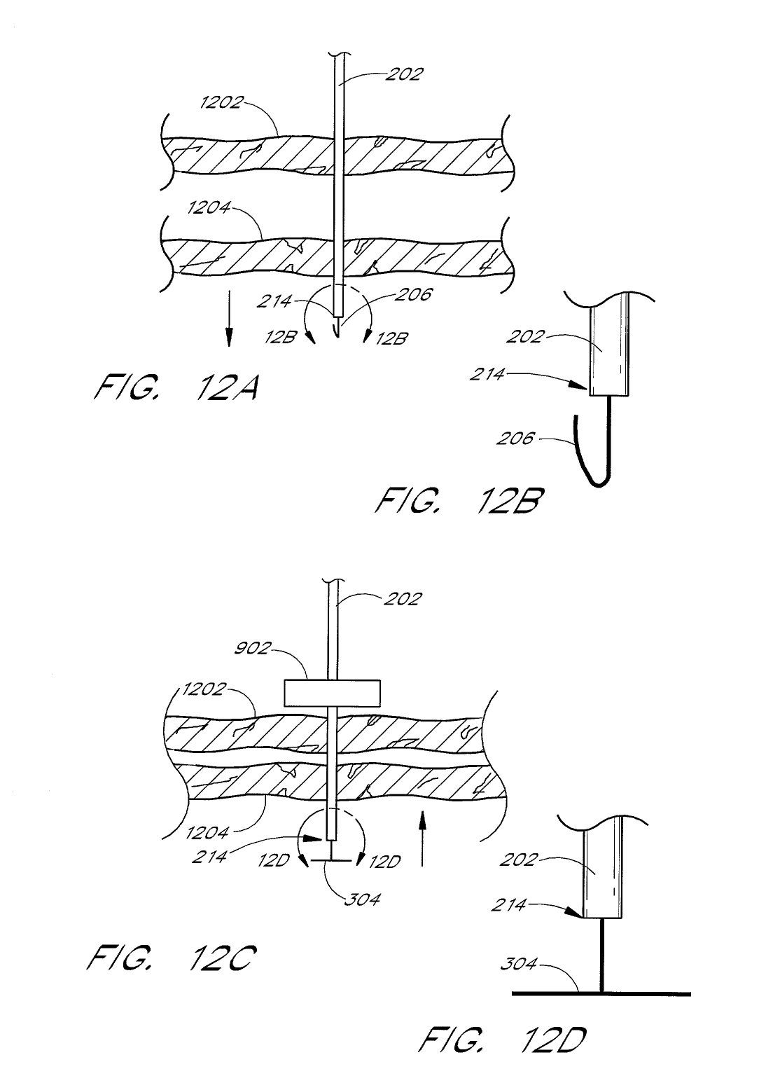

With reference now to FIGS. 12A-12D, a first tissue 1202 is illustrated adjacent a second piece of tissue 1204. The distal tip 214 of the suture sheath 202 penetrates the tissues 1202 and 1204. The suture 204 or 302 is advanced through the suture sheath 202 so that the suture hook (J-hook 206 or T-bar 304) is expelled from the tip 214 of the suture sheath 202. The suture hook opens at the distal side of the second piece of tissue 1204. The suture is then pulled back against the tissue 1204 so that the deployed suture hook is pulled tight against the wall of the tissue 1204. The clip 902 is then applied to the suture at the proximal side of the first tissue 1202 to secure the suture and to hold the tissues 1202 and 1204 together.

The applicator 602 allows one-handed suturing. The applicator 602 is positioned so that the suture sheath 202 penetrates the tissue to be sutured. The flange 620 regulates the penetration depth of the suture sheath 202 into the tissue. The safety lock 412 is then removed and the proximal handle 604 is squeezed towards the distal handle 606, thereby expelling the J-hook 206 or T-bar 304 suture hook from the suture sheath 202. Upon expulsion from the suture sheath 202, the J-hook 206 or T-bar 304 suture hook is deployed. When tension is applied to the suture, the J-hook 206 or T-bar 304 suture hook prevents the suture from being pulled back through the entry hole. The suture applicator 602 is then withdrawn from the tissue.

While maintaining pressure on the suture with one hand, the surgeon uses the other hand to grip the suture clip applicator 1002 and to guide its tip to the location of the suture protruding from the tissue. The surgeon forces the bottom plane of the suture clip snug against the tissue, to create a pressure sufficient to successfully join one tissue to another. Those of ordinary skill will appreciate that the amount of such pressure is known in the art and differs from procedure to procedure.

The surgeon then applies pressure to the lever 1004, pushing it towards the handle 1006. This causes the wedge 914 to move upwardly and to become dislodged from the bore 908 of the suture clip 902. The suture clip wings 904 and 906 then resiliently close onto and firmly grasp the suture 204 or 302. The surgeon may then cut the suture above the clip 902 to complete one stitch.

When an endoscopic end-to-side vascular anastomosis is performed, a tubular vascular graft, either natural (which may be allogeneic or autologous) or artificial (such as a prosthetic graft made of Gore-Tex), is anastomosed to the side of an artery by repeating the above suturing method around the periphery of the graft. After the graft is sutured to the side wall of the artery, it is tested for leaks by introducing normal saline into the graft under pressure. If there is a leak, an additional stitch is placed at the location of the leak. If no leak is present, the side wall of the artery may be cored out inside the anastomosed graft, establishing continuity between the arterial lumen and the graft.

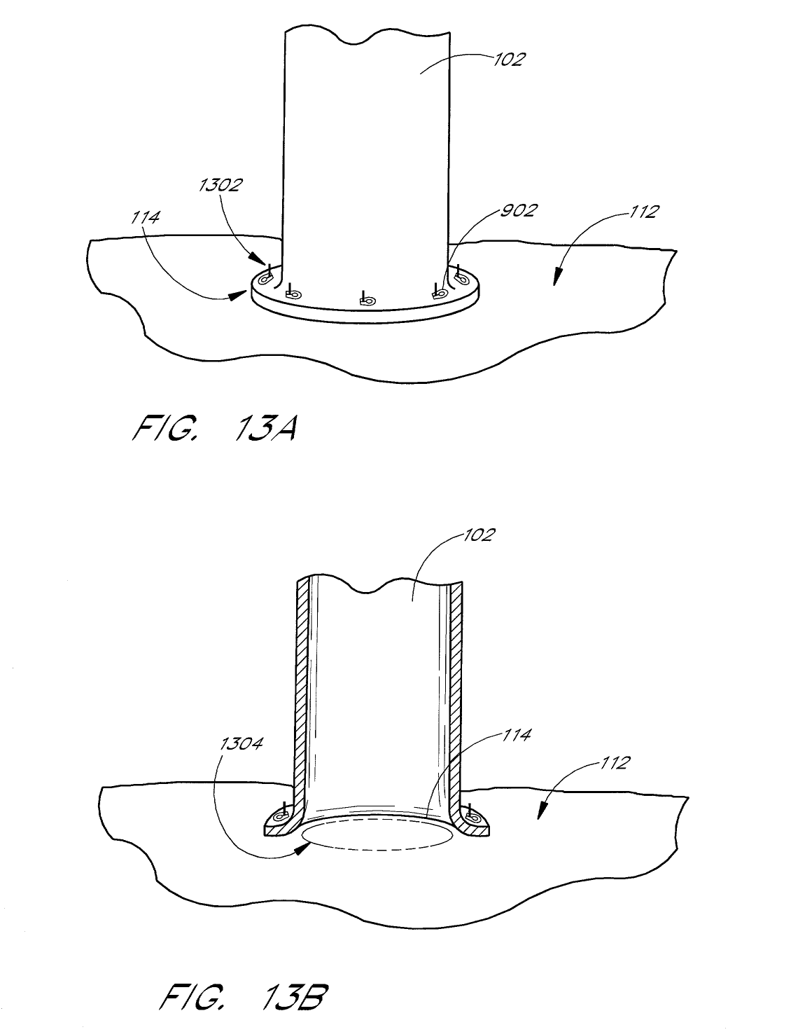

FIG. 13A illustrates a view of an arterial graft anastomosis performed in accordance with the present invention. Thus, an artery or prosthetic graft 102 is sutured to a piece of tissue 112. The side wall of the artery 1302 is held snug against the tissue 112 with a number of stitches, each fixed with a suture clip 902.

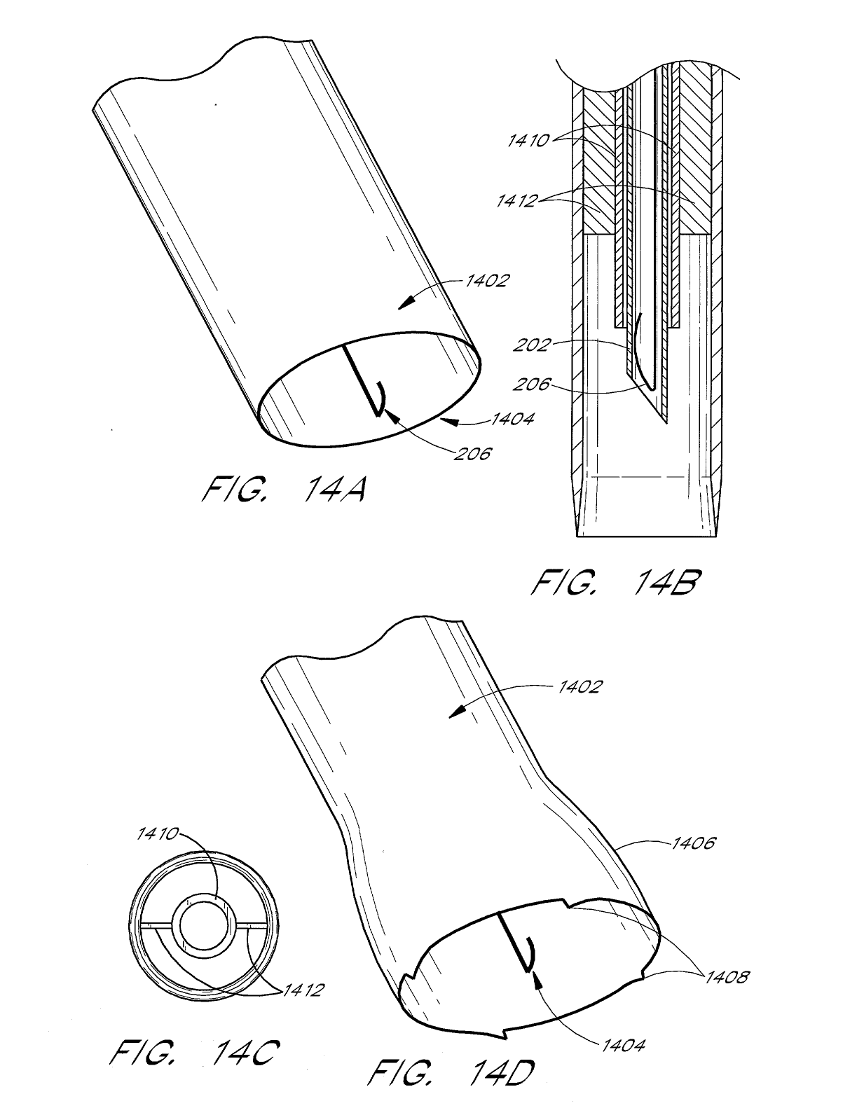

FIG. 13B is a cross-sectional view of an arterial graft. The material to be removed 1304 (indicated by a dashed line) is interior to and circumscribed by the anastomotic line 114. Such arteriotomy can performed by known techniques. However, a heretofore unknown and preferred technique for performing the arteriotomy is illustrated in FIG. 14.

As illustrated in FIG. 14A, an arteriotomy tool 1402 comprises a tubular body having a sharpened distal end 1404. The tool 1402 is configured to provide for deployment of a J-hook 206 or a similar hook at the distal end. The hook 206 holds the portion of tissue to be removed from the artery, allowing that portion to be withdrawn upon completion of cutting.

The tool 1402 is advanced through the arterial graft until the hook 206 penetrates the side wall of the host artery and the sharpened distal end 1404 of the tool 1402 contacts the side wall of the artery. The tool 1402 is then rotated axially to core through the arterial wall. The hook 206 remains attached to the cored out material, which can then be removed through the graft artery along with the tool 1402. In one embodiment the hook 206 may be fixed to be axially centered within the tool body such that it protrudes from the distal end a distance sufficient to penetrate the tissue to be removed. Such fixing of the hook 206 may be accomplished using a variety of means, including any non-toxic cement.

In another embodiment of the arteriotomy tool 1402, as illustrated in the lateral cross-sectional view of FIG. 14B and the frontal cross-sectional view of FIG. 14C, a tubular guide channel 1410 is attached to and centered axially within the body of the tool 1402 by centering members 1412. The guide channel 1410 runs along the length of the body of the tool 1402, stopping short of the distal end 1414, thus allowing sufficient room within the distal end of the body to receive a removed piece of tissue therein.

Preferably, a suture sheath 202 (as described above) carries a J-hook suture hook 206 inside its distal end. The suture sheath 202 is inserted into the guide channel 1410 so that its distal end rests within the distal end of the tool body 1402.

With an arterial graft stitched in place, the arteriotomy tool 1402 is inserted into the open end of the graft until it meets the tissue to be removed. The suture sheath 202 is then advanced to penetrate the tissue to be removed. The suture hook 206 is then preferably deployed by using the handle assembly described in connection with FIGS. 2A, 2B, 2C, 2D, 4A, 4B, 4C, 4D, 5A and 5B and optionally the applicator described in connection with FIG. 6. With the suture hook 206 in place, the tool 1402 may be rotated to cut a circular piece of tissue from the artery, and the tissue piece may then be removed along with the arteriotomy tool 1402 and handle assembly.

In another embodiment, the hook 206 may be fixed to a substantially rigid shaft (not shown), the shaft and hook 206 introduced into the guide channel 1410, with the tool 1402 in place in the graft, until the hook penetrates the tissue to be removed. At that point, the surgeon may pull back slightly on the shaft to engage the hook into the tissue to be removed, whereupon coring may be performed by rotating the tool 1402. When coring is complete, the tool 1402, along with the shaft, hook 206 and severed tissue may be removed from the graft.

In still another embodiment illustrated in FIG. 14D, the arteriotomy tool 1402 has a larger diameter around the body 1406 of the distal end 1404, that diameter sized to closely match the internal diameter of the graft vessel. Thus, friction caused by contact of the outer wall of the tool with the inner wall of the graft vessel may advantageously be minimized for easier rotation during the coring operation. Also advantageously, the sharpened rim of the distal end 1404 of the tool 1402 may bear one or more serrated teeth 1408, as illustrated in FIG. 14D, to improve cutting during coring.

The illustrated suturing device and method for perpendicular suturing allows suturing to be performed in a restricted space or when a conventional rotational movement of a needle holder is not feasible. For instance, the vascular clamps required for traditional suturing techniques interfere with the maneuvering of endoscopic surgical instruments. Two endoscopic surgical instruments cannot easily be inserted at right angles to one another into a body cavity for suturing. The disclosed apparatus and methods address these problems, among others.

The invention can also be used in end-to-side anastomoses of other tubular structures, such as the esophagus, stomach, small intestine, large intestine, colon, ureters, billiary ducts, pancreatic duct, and bladder. The invention is also useful in artery-to-vein, vein-to-vein and artery- or vein-to-graft anastomoses.

The invention may be embodied in other specific forms without departing from the essential characteristics described herein. The embodiments described herein are to be considered in all respects as illustrative only and not restrictive in any manner. The scope of the invention is indicated by the following claims rather than by the foregoing description. Any and all modifications which come within the meaning and range of equivalency of the claims are to be considered within their scope.

What is claimed is:

1. A suturing device, comprising:
a sheath having a proximal end and a distal end, said distal end configured to enter soft tissue when moved in a first direction;
a sheath handle at said proximal end of said sheath;
a suture slidably disposed within said sheath and having a proximal end and a distal end;
a suture handle at said proximal end of said suture, said suture handle configured to move said suture within said sheath when moved relative to said sheath handle; and
a suture hook at said distal end of said suture, said suture hook within said sheath when said suture handle is located a first distance from said sheath handle, said suture hook expelled from said sheath when said suture handle is positioned to a second distance from said sheath handle, said suture hook deploying upon said expelling to resist movement of said suture in a second direction substantially opposite said first direction.

2. The suturing device as described in claim 1, wherein said suture hook is a J-hook suture hook.

3. The suturing device as described in claim 1, wherein said suture hook is a T-bar suture hook.

4. The suturing device as described in claim 1, wherein said suture hook is integrally formed with said suture distal end.

5. The suturing device as described in claim 1, wherein said suture hook is selectively attachable to said suture distal end.

6. The suturing device as described in claim 1, wherein said suture is a wire suture.

7. The suturing device as described in claim 1, wherein said suture is a braided suture.

8. The suturing device as described in claim 1, wherein said suture is a monofilament suture.

9. The suturing device as described in claim 1, further comprising:
a suture applicator having first and second applicator members, said suture applicator configured to move said suture handle relative to said suture sheath handle when said first and second applicator members are moved relative to each other.

10. The suturing device as described in claim 9, wherein said suture applicator is operable with one hand.

11. The suturing device as described in claim 9, wherein said suture applicator facilitates said expelling of said suture hook and said first and second directions are substantially perpendicular to a surgical field.

12. The suturing device as described in claim 9, further comprising:
a flange on a distal end of said suture applicator to regulate a penetration of said sheath within said soft tissue.

13. The suturing device as described in claim 9, further comprising:
a lock substantially preventing movement of the suture sheath handle relative to the suture handle.

14. A suture clip applicator, comprising:
a shaft with a handle;
a triggering member; and
a clip wedge that moves relative to said shaft and dislodges from a suture clip upon an activation of said triggering member.

15. A suture clip applicator, comprising:
a shaft having a clip retention member;
a lever movably coupled to said shaft; and
a clip wedge linked to said lever so that said clip wedge moves relative to said clip retention member when said lever is moved.

16. The suture clip applicator described in claim 15, further comprising:
a suture clip having two wings and a wedge bore, said wings biased toward each other, said suture clip configured to receive said clip wedge in said wedge bore to separate said wings by a distance greater than a suture diameter, said clip retention member configured to hold said suture clip substantially stationary while said clip wedge moves, said holding of said suture clip causing said clip wedge to be withdrawn from said wedge bore whereupon said wings move toward each other.

17. The suture clip applicator as described in claim 16, wherein said suture clip applicator positions said suture clip to a position along a suture wherein, prior to said withdrawing of said clip wedge, said suture is between said wings and said suture clip is proximate a soft tissue undergoing suturing, and wherein said shaft is substantially perpendicular to a surgical site during said positioning and during said withdrawing of said clip wedge.

18. The suture clip applicator as described in claim 17, wherein said suture clip applicator is adapted so that one hand can perform both said positioning and said withdrawing.

19. The suture clip applicator as described in claim 18, wherein said suture clip is configured so that said wings grip a suture between them with a first force sufficient to maintain said grip in the presence of a second force applied to said suture clip in a direction along said suture, said second force substantially the same as that required to hold two to tissues together in a successful surgical suturing procedure.

20. A suture clip applicator, comprising:
an applicator frame having an applicator shaft;
an applicator handle connected to said applicator shaft near a handle end of the applicator shaft;
a shoulder connected to said applicator shaft near a clip end of the applicator shaft;
a lever movably coupled to said applicator frame;
a retractor coupled to said lever, said retractor configured to move from an apply position into a retract position in a path substantially along said applicator shaft when said lever is moved relative to said applicator handle;
a clip wedge connected to a clip end of said retractor; and
a suture clip having opposing wings biased to exert a force toward each other, said clip wedge fitted into a wedge bore of said suture clip when said retractor is in said apply position, said fitting of said clip wedge creating a distance between said wings that is greater than the diameter of a suture, said shoulder preventing said suture clip from travelling with said clip wedge in said path when said lever is moved relative to said applicator handle, said movement of said lever thus removing said clip wedge from said wedge bore and causing said wings of said suture clip to spring together.

21. A method for suturing, said method comprising the steps of:
(1) advancing a suture assembly into a surgical field in an operating direction substantially perpendicular to said surgical field until a distal end of a suture sheath of said suture assembly penetrates tissues to be sutured together;
(2) moving a suture within said suture sheath such that a suture hook on a distal end of said suture emerges from the distal end of said suture sheath and deploys to resist withdrawal of said suture from said penetrated tissues; and
(3) removing said suture assembly from said surgical field in a direction substantially opposite said operating direction while said suture hook remains deployed with said suture attached thereto.

22. The method for suturing described in claim 21, further comprising the steps of:
(4) advancing a suture clip applicator into said surgical field in said operating direction;
(5) positioning a suture clip carried by said suture clip applicator about said suture at a position along said suture appropriate to create a pressure upon said tissues at least sufficient to surgically join said tissues;
(6) releasing said suture clip such that it grips said suture to maintain said pressure; and
(7) withdrawing said suture clip applicator from said surgical field in a direction substantially opposite said operating direction.

23. The method for suturing described in claim 22, wherein multiple stitches of a surgical suturing procedure are placed in tissues by repeating steps (1) through (7).

24. The method for suturing described in claim 22, said method comprising the further steps of:
placing a clip wedge between two wings of said suture clip prior to said positioning of said suture clip; and
removing said clip wedge during said releasing of said suture clip.

25. The method for suturing described in claim 21, wherein said advancing of said suture assembly and said moving of said suture within said suture sheath are both performed by operating a suture applicator with a single hand, said suture applicator carrying said suture assembly.

26. A method for suture clipping, said method comprising the steps of:
advancing a suture clip applicator into a surgical field in an operating direction substantially perpendicular to said surgical field;
positioning a suture clip carried by said suture clip applicator about said suture at a position along said suture appropriate to create a pressure at least sufficient to surgically join said tissues; and
releasing said suture clip such that it grips said suture to maintain said pressure.

27. The method for suture clipping described in claim 26, the method comprising the further steps of:
placing a clip wedge between two wings of said suture clip prior to said positioning of said suture clip; and
removing said clip wedge during said releasing of said suture clip.

28. The method for suture clipping described in claim 27, wherein said advancing, said positioning, said removing and said releasing are all performed by operating said suture clip applicator with one hand.

29. An arteriotomy tool comprising:
a tubular body, said distal end of said tubular body having a sharpened edge to cut through and sever tissue upon rotation of said tubular body, and
a hook provided at said distal end of said tubular body to hold said severed tissue.

30. The arteriotomy tool as described in claim 29 wherein said hook is provided through a guide channel within said tubular body.
